(19) United States
(12) Patent Application Publication
(10) Pub. No.: US 2020/0269865 A1
(43) Pub. Date: Aug. 27, 2020

(54) METHOD OF RECOGNIZING EMOTION OF DRIVER AND APPARATUS USING THE SAME

(71) Applicant: ELECTRONICS AND TELECOMMUNICATIONS RESEARCH INSTITUTE, Daejeon (KR)

(72) Inventors: Chang Rak YOON, Daejeon (KR); Young Jin KWON, Daejeon (KR); Kyong Ho KIM, Daejeon (KR); Do Hyun KIM, Daejeon (KR); Whui KIM, Ulsan (KR); Kyong Hee LEE, Daejeon (KR); Byung Tae JANG, Daejeon (KR); Ju Il JEON, Daejeon (KR); Jin Kyu CHOI, Daejeon (KR); Hyun Kyun CHOI, Ulsan (KR); Yoon Sook HWANG, Daejeon (KR)

(21) Appl. No.: 16/790,480

(22) Filed: Feb. 13, 2020

(30) Foreign Application Priority Data
Feb. 21, 2019 (KR) 10-2019-0020798

(51) Int. Cl. B60W 50/14; G10L 25/63; G06K 9/00; A61B 5/16; A61B 5/18; B60W 40/08

(52) U.S. Cl. CPC B60W 50/14; G10L 25/63; G06K 9/00845; A61B 5/02055; A61B 5/165; A61B 5/18; B60W 40/08; G06K 9/00315

(57) ABSTRACT

Provided are a method of recognizing an emotion of a driver and an apparatus using the same. The method includes receiving emotion recognition information including at least one of vehicle state information and driver emotion state information, determining whether a dominant emotion state exists among one or more emotion states possessed by the driver on the basis of the emotion recognition information and an emotion recognition model, and performing a reaction inducing interaction with respect to the driver determined on the basis of the determined dominant emotion state together with the driver when the dominant emotion state of the driver is determined to exist.

### CROSS-REFERENCE TO RELATED APPLICATION

This application claims priority to and the benefit of Korean Patent Application No. 2019-0020798, filed on Feb. 21, 2019, the disclosure of which is incorporated herein by reference in its entirety.

### BACKGROUND

### 1. Field of the Invention

The present invention relates to a method of recognizing an emotion of a driver and an apparatus using the same, and more specifically, to a method for improving the accuracy in recognizing an emotion state of a driver by performing a reaction inducing interaction with the driver on the basis of emotion information of the driver and vehicle state information and checking a reaction corresponding to the interaction, and an apparatus using the same.

### 2. Discussion of Related Art

While the number of vehicle users is drastically increasing, road construction enlargement cannot keep up with the increasing number of vehicle users, thereby causing traffic congestion to become worse day by day. In such a situation, the drivers are subject to various stressful conditions during driving, which may inhibit safe driving of the driver. In addition, with population aging, the number of traffic accidents caused by driving of elderly drivers is increasing.

Meanwhile, there has been growing attention on an emotion recognition technology for measuring an emotion (including a sensibility) of a human. The emotion recognition technology is provided to recognize an emotion of a human through an analysis of a facial expression, an analysis of an eyeball movement, an analysis of language used, an analysis of various biosignals of a human body, and the like.

In addition, there is an expectation about a great demand for a semi-self driving vehicle based on involvement of a human driver before development and propagation of full-self driving vehicle. In view of the above-described circumstances, there is a need to increase the driver's safety through rapid and accurate recognition of an emotion state of a driver (hereinafter, the driver of the embodiments means a human driver).

Meanwhile, an emotion felt by a driver is caused by a complex driving environment, so it is difficult to assert that the same emotion is felt by a specific condition. Accordingly, there is a need to continuously update an emotion recognition method and an interaction method according to circumstances.

However, the conventional technology of recognizing an emotion of a driver mostly recognizes an emotion of a driver according to a previously trained emotion recognition apparatus and method and provides the driver with feedback according to a predefined interaction apparatus and method. In this case, the feedback must be provided depending on the previously trained emotion recognition method while being restricted by the predefined interaction method. In addition, it is difficult to ensure sufficient reliability in the driver emotion recognition, which requires a high level of reliability in association with safety.

### SUMMARY OF THE INVENTION

The present invention is directed to providing a method of identifying an emotion state of a driver on the basis of emotion information of the driver and vehicle state information, and an apparatus using the same.

The present invention is directed to providing a method of rapidly and accurately identifying an unclear emotion state of a driver through a reaction inducing interaction process between a driver and a vehicle, and an apparatus using the same.

The technical objectives of the present invention are not limited to the above, and other objectives may become apparent to those of ordinary skill in the art based on the following descriptions.

According to an aspect of the present invention, there is provided a method of recognizing an emotion of a driver, the method including receiving emotion recognition information including at least one of vehicle state information and driver emotion state information, determining whether a dominant emotion state exists among one or more emotion states possessed by the driver on the basis of the emotion recognition information and an emotion recognition model, and performing a reaction inducing interaction with respect to the driver determined on the basis of the determined dominant emotion state together with the driver when the dominant emotion state of the driver is determined to exist.

The determining of whether the dominant emotion state exists may include identifying the number of emotion states possessed by the driver from the emotion recognition information, converting the identified emotion states into dominant emotion indexes, identifying whether a dominant emotion index exceeding a predetermined threshold value among the dominant emotion indexes exists, and in response to an existence of the dominant emotion indexes exceeding a predetermined reference value, determining an emotion state corresponding to a dominant emotion index exceeding the predetermined reference value to a largest degree to be the dominant emotion state.

The performing of the reaction inducing interaction with respect to the driver together with the driver may include requesting the driver to perform a specific action related to a drive control, receiving driver feedback information responsive to the request for performing the specific action from the driver, adjusting a weight of the reaction inducing interaction upward when the received driver feedback information is determined to be a positive response, and adjusting a weight of the reaction inducing interaction downward when the received driver feedback information is determined to be a negative response.

The adjusting of the reaction inducing interaction upward may include at least one of adjusting a weight of a conversion of a dominant emotion index upward in the determining of whether the dominant emotion state exists and adjusting a weight of the request for the performance of the specific action upward in the performing of the reaction inducing interaction with the driver.

The adjusting of the reaction inducing interaction downward may include at least one of adjusting a weight of a conversion of a dominant emotion index downward in the determining of whether the dominant emotion state exists and adjusting a weight of the request for the performance of the specific action downward in the performing of the reaction inducing interaction with the driver.

The method may further include, when the dominant emotion state of the driver is determined to not exist, selecting N emotion states starting from an emotion state having a highest dominant emotion index first among the one or more emotion states derived in the determining of whether the dominant emotion state of the driver exists, determining an emotion state randomly selected from the N emotion states to be a reserved dominant emotion state, and performing a reaction inducing interaction with respect to the driver determined on the basis of information about the determined reserved dominant emotion state together with the driver.

The adjusting of a weight of the reaction inducing interaction downward may include changing the request for performing the specific action with respect to the dominant emotion state of the driver when the dominant emotion state of the driver is determined to exist and driver feedback information indicates a negative response and updating the determined reserved dominant emotion state when the dominant emotion state of the driver is determined to not exist and the driver feedback information indicates a negative response.

According to another aspect of the present invention, there is provided an apparatus for recognizing an emotion of a driver, the apparatus including an emotion recognition information input unit configured to receive emotion recognition information including at least one of vehicle state information and driver emotion state information, an emotion recognition model storage unit configured to store an emotion recognition model of the driver, a driver emotion recognition unit configured to generate at least one of dominant emotion state information and reserved dominant emotion state information of the driver on the basis of the received emotion recognition information and the emotion recognition model, an interaction determination unit configured to request the driver to perform a specific action related to a drive control on the basis of the determined at least one of the dominant emotion state information and the reserved dominant emotion state information of the driver, a driver response measurement unit configured to receive driver feedback information responsive to the request for performing the specific action from the driver, and a recognition information storage unit configured to store at least one of the emotion recognition information of the driver, the dominant emotion state information of the driver, and the reserved dominant emotion state information of the driver.

The apparatus may further include an emotion recognition model update unit configured to perform an update on the emotion recognition model on the basis of information about updating the emotion recognition model of the driver that is generated on the basis of the driver feedback information.

The driver emotion recognition unit may be configured to identify the number of emotion states possessed by the driver from the emotion recognition information, convert the identified emotion states into dominant emotion indexes, identify whether a dominant emotion index exists that exceeds a predetermined threshold value among the dominant emotion indexes, and in response to an existence of the dominant emotion indexes exceeding a predetermined reference value, generate an emotion state corresponding to a dominant emotion index exceeding the predetermined reference value to a largest degree as dominant emotion state information.

The driver emotion recognition unit may be further configured to identify the number of emotion states possessed by the driver from the emotion recognition information, convert the identified emotion states into dominant emotion indexes, identify whether a dominant emotion index exists that exceeds a predetermined threshold value among the dominant emotion indexes, sequentially select N dominant emotion indexes starting from a largest dominant emotion index first among the dominant emotion indexes in response to an absence of the dominant emotion index exceeding a predetermined reference value, and generate an emotion state associated with a dominant emotion index randomly selected among the N dominant emotion indexes as reserved dominant emotion state information.

The driver response measurement unit may adjust a weight of the request sent to the driver for the performance of the specific action related to the drive control upward when the received driver feedback information is determined to be a positive response, wherein the adjusting of the weight upward may include at least one of adjusting a weight of the generation of the dominant emotion state information performed by the driver emotion recognition unit upward and adjusting a weight of the request for the performance of the specific action sent by the interaction determination unit upward.

The driver response measurement unit may adjust a weight of the request sent to the driver for the performance of the specific action related to the drive control downward when the received driver feedback information is determined to be a negative response, wherein the adjusting of the weight downward may include at least one of adjusting a weight of the generation of the reserved dominant emotion state information performed by the driver emotion recognition unit downward and adjusting a weight of the request for the performance of the specific action sent by the interaction determination unit downward.

The downward adjusting of the weight of the request for the performance of the specific action sent by the interaction determination unit may include when the driver response measurement unit receives negative driver feedback information in response to a request sent by the interaction determination unit to the driver for performing a drive-control related specific action based on the dominant emotion state information, changing the request for performing the drive-control related specific action associated with the dominant emotion state information of the driver, and when the driver response measurement unit receives negative driver feedback information in response to a request sent by the interaction determination unit to the driver for performing a drive-control related specific action based on the reserved dominant emotion state information, updating the generated reserved dominant emotion state information.

According to another aspect of the present invention, there is provided a method of recognizing an emotion of a driver, the method including receiving emotion recognition information including at least one of vehicle state information and driver emotion state information, deriving at least one emotion state of the driver included in the emotion recognition information, converting the derived emotion states of the driver into dominant emotion indexes, arranging the converted dominant emotion indexes in an order of magnitude, and determining whether a dominant emotion exists by comparing the arranged dominant emotion indexes with a reference value for determining the dominant emotion.

The determining of whether the dominant emotion exists may include: deriving a dominant emotion index larger than the reference value, and in response to an existence of one or more dominant emotion indexes larger than the reference value, selecting a largest dominant emotion index and determining a dominant emotion state associated with the largest dominant emotion index to be a dominant emotion state.

The determining of whether the dominant emotion exists may include deriving a dominant emotion index larger than the reference value, sequentially selecting N dominant emotion indexes starting from a largest dominant emotion index first in response to an absence of the dominant emotion index larger than the reference value, and determining an emotion state associated with a dominant emotion index randomly selected among the N dominant emotion indexes to be a reserved dominant emotion state.

### BRIEF DESCRIPTION OF THE DRAWINGS

FIG. 1A and FIG. 1B are flowcharts for describing a method of recognizing an emotion of a driver according to an embodiment of the present invention.

FIG. 2 is a flowchart for describing a method of determining a dominant emotion state and a reserved dominant emotion state according to an embodiment of the present invention.

FIG. 3 is a block diagram for describing an apparatus for recognizing an emotion of a driver according to an embodiment of the present invention.

### DETAILED DESCRIPTION OF EXEMPLARY EMBODIMENTS

While the present invention is susceptible to various modifications and alternative embodiments, specific embodiments thereof are shown by way of example in the accompanying drawings and will be described. However, it should be understood that there is no intention to limit the present invention to the particular embodiments disclosed, but on the contrary, the present invention is to cover all modifications, equivalents, and alternatives falling within the spirit and scope of the present invention.

It will be understood that, although the terms first, second, etc. may be used herein to describe various elements, the elements should not be limited by the terms. The terms are only used to distinguish one element from another. For example, a first element could be termed a second element, and, similarly, a second element could be termed a first element without departing from the scope of the present invention. As used herein, the term “and/or” includes any one or combination of a plurality of the associated listed items.

It will be understood that when an element is referred to as being “connected” or “coupled” to another element, it can be directly connected or coupled to another element or intervening elements may be present. In contrast, when an element is referred to as being “directly connected” or “directly coupled” to another element, there are no intervening elements present.

The terminology used herein is for the purpose of describing particular embodiments only and is not intended to be limiting of the present invention. As used herein, the singular forms “a,” “an,” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components but do not preclude the presence or addition of one or more other features, integers, steps, operations, elements, components, and/or groups thereof

Unless otherwise defined, all terms including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, example embodiments of the present invention will be described with reference to the accompanying drawings in detail. For better understanding of the present invention, the same reference numerals are used to refer to the same elements through the description of the figures, and the description of the same elements will be omitted.

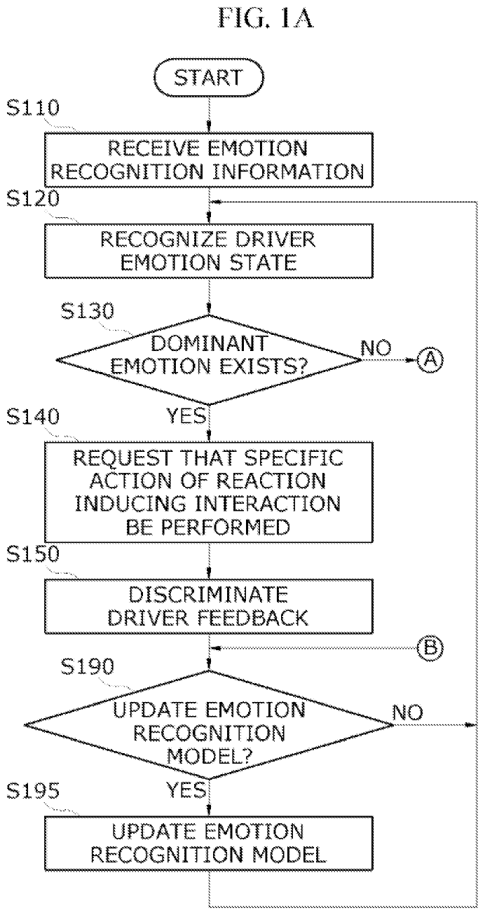

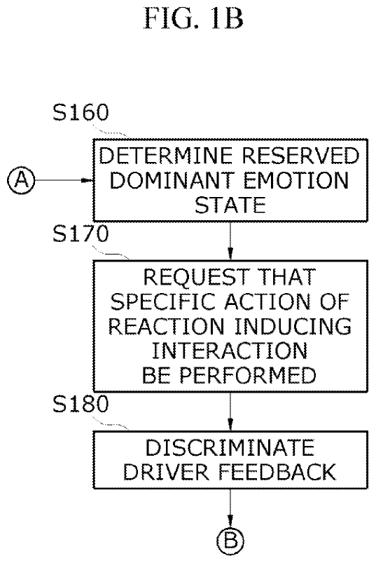

FIG. 1A and FIG. 1B are flowcharts for describing a method of recognizing an emotion of a driver according to an embodiment of the present invention.

Referring to FIG. 1A, the method of recognizing an emotion of a driver according to the embodiment of the present invention first includes acquiring emotion recognition information including information about an emotion state of a driver (referred to as driver emotion state information) and information about a state of a vehicle driven by the driver (referred to as vehicle state information) (S110).

The driver emotion state information includes a facial expression, dialogue content, an action (including a gesture), and biosignals, such as a blood pressure and a body temperature, of a driver. The driver emotion state information may be measured using sensors installed in the vehicle (a camera, an image sensor, a temperature sensor, and the like.) The vehicle state information is information related to the driving of the vehicle such as a speed, a degree of acceleration/deceleration, a rotation state, sudden acceleration, internal and external lighting conditions, and a noise state of the vehicle. Such vehicle state information may be measured from various sensors installed in the vehicle or received through an in-vehicle device such as an electronic control unit (ECU).

Referring to FIG. 1A, on the basis of a result of comparing the acquired emotion recognition information with a previously provided emotion recognition model, the emotion state of the driver is recognized (S120). One or more emotion states considered to be possessed by the driver may be recognized through the driver emotion state recognition. That is, the driver may be currently recognized to have an emotion of joy, an emotion of sadness, an emotion of tiredness, or an emotion of being eager to take a rest.

Referring to FIG. 1A, the method of recognizing the emotion of the driver according to the present invention determines an existence of a dominant emotion state, which is considered a current main emotion, of the driver on the basis of the comparison of the emotion recognition information and the emotion recognition model (S130). In order to determine the existence of the dominant emotion state, it is determined whether an emotion state exceeding a predetermined reference value (a threshold value) exists (i.e., when two or more emotion states exceed the predetermined reference value, an emotion state having a larger value is identified to be the dominant emotion state. The method of comparing the emotion state with the reference value will be described in detail below.)

Referring to FIG. 1B, when the emotion state exceeding the predetermined reference value is determined to not exist as a result of determining whether the dominant emotion exists (i.e., a state of the recognition being reserved), a reserved dominant emotion state is determined (S160). The reserved dominant emotion state refers to an emotion state that does not correspond to a dominant emotion state but is temporarily set as a dominant emotion state among a plurality of recognized emotion states so as to perform a subsequent reaction inducing interaction procedure, and details thereof will be described below.

Through the procedure as such, the dominant emotion state or the reserved dominant emotion state is determined, and a reaction inducing interaction for performing a specific action suitable for the corresponding emotion state is performed. The reaction inducing interaction represents an interaction between a driver and an emotion recognition apparatus performing the method of recognizing the emotion of the driver according to the embodiment of the present invention, in which the emotion recognition apparatus identifies an emotion of the driver (a dominant emotion state or a reserved dominant emotion state), requests that a drive control-related specific action associated with the emotion of the driver should be performed (induction of a reaction), and receives feedback (performance of the requested specific action by the driver) from the driver.

That is, the reaction inducing interaction refers to a process of requesting that a drive control-related specific action with respect to a driver emotion state should be performed. The specific action requested by the emotion recognition apparatus to the driver is an action expected to be positively performed by the driver receiving the request regardless of whether the driver recognizes his or her own emotion state.

That is, when the driver is identified to have an emotion state of distraction, the emotion recognition apparatus may request to the driver to control the speed adjustment, the lighting adjustment, and the volume adjustment of the vehicle. In this regard, the driver may perform the actions as requested or may not perform the actions. Whether the requested action has been performed as requested is referred to as a driver feedback. Such a process of requesting that a specific action be performed with respect to an identified (dominant) emotion state of the driver and receiving a positive drive feedback in response to the request is referred to as a reaction inducing interaction.

The reaction inducing interaction according to the embodiment of the present invention includes an explicit reaction inducing interaction in which an explicit positive feedback is expected from the driver and a potential reaction inducing interaction in which an implicit positive feedback is required from the driver. An example of the explicit reaction inducing interaction includes, when a dominant emotion state of a driver is identified as anger, sending the driver an explicit request for a deceleration, receiving an explicit positive response (e.g., “yes” in speech), and performing the deceleration. An example of the potential reaction inducing interaction includes, when a dominant emotion state of a driver is identified as anger, sending the driver a request for performing a deceleration action, and performing, by the driver, the deceleration in response to the request.

The method of recognizing the emotion of the driver according to the embodiment of the present invention may allow a drive control-related specific action to be performed with respect to a normal emotion state of a driver. Table 1 shows an example of a mapping relationship between normal emotion states and drive control-related specific action executions according to an embodiment of the present invention (but, the present invention is not limited thereto and may include performing specific actions that are divided in a larger number according to various emotions.)

TABLE 1

| | Drive-control related specific action in | |
|---|---|---|
| Driver emotion state | reaction inducing interaction | |
| (dominant/reserved) | Current status | Change needed status |
| Anger | Speeding | Decelerate by 20 km |
| Distraction | Headlights turned | Turn on front headlights |
| | off (night) | |
| | Loud music | Turn music volume down |
| Tiredness | Speeding | Decelerate by 10 km |
| | Driving | Move forward 5 km to |
| | | rest stop to rest |
| . . . | . . . | . . . |

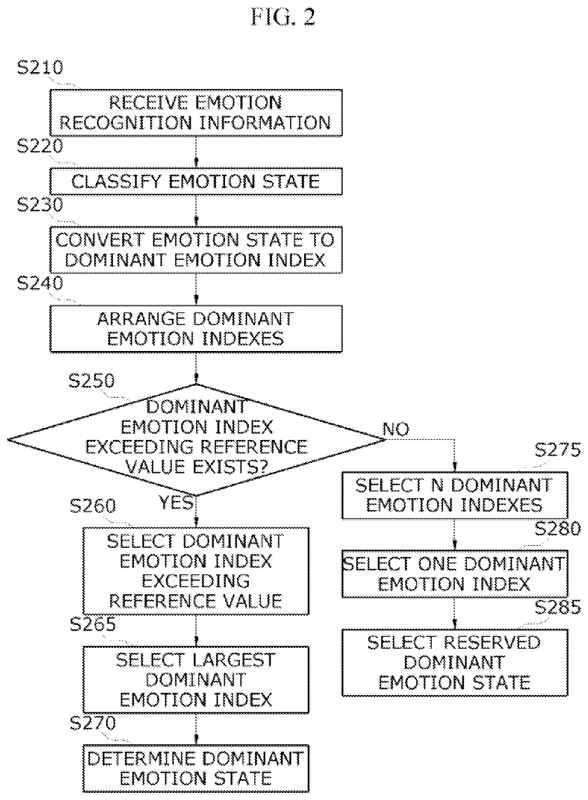

Meanwhile, the determining of the dominant emotion state and the reserved dominant emotion state in the method of recognizing the emotion of the driver may be performed by a method shown in FIG. 2.

First, the emotion recognition information in the method of recognizing the emotion of the driver described with reference to FIG. 1A and FIG. 1B is received (S210). When the emotion recognition information is analyzed, one or more emotion states may be derived. The derived emotion states are classified according to types of emotion state (S220). The classified emotion states are converted to dominant emotion indexes (S230). An example of the conversion of the dominant emotion index includes identifying the intensity of the derived emotion state and converting the intensity into a quantification value or a level (e.g., strong, medium, weak). When one or more emotion states are derived, the derived emotion states are arranged on the basis of the magnitudes of the dominant emotion indexes thereof (S240). The dominant emotion indexes arranged on the basis of the magnitudes thereof are compared with a predetermined reference value (a threshold value) and whether a dominant emotion index exceeding the reference value exists is identified (S250).

When a dominant emotion index exceeding the reference value exists, the dominant emotion index is selected (S260). When two or more dominant emotion indexes exceed the reference value, the largest of the dominant emotion indexes is selected (S265). An emotion state having the largest dominant emotion index is determined to be the dominant emotion state (S270).

A state in which a dominant emotion index exceeding the reference value does not exist or it is difficult to infer a single type of dominant emotion state since two or more emotions having the same level are mixed is classified as a state of a dominant emotion recognition being reserved. However, even in the state of a dominant emotion recognition being reserved, it is preferable to perform the following operations (S275 to S285) because the reaction inducing interaction is applicable to the state of a dominant emotion recognition being reserved, and the dominant emotion state may be inferred through the reaction inducing interaction.

That is, when a dominant emotion index exceeding the reference value does not exist, top N (for example, N=3) dominant emotion indexes are selected on the basis of the magnitudes of the dominant emotion indexes (S275). One of the selected N dominant emotion indexes is selected (S280). A criterion for the selection may include selecting the largest dominant emotion index, selecting an average dominant emotion index among selected dominant emotion indexes, selecting a random dominant emotion index, and other various methods. The emotion state corresponding to the selected dominant emotion index is determined to be the reserved dominant emotion state (S285).

When a dominant emotion state is determined to exist as a result of determining whether the dominant emotion state exists through the above process, an explicit/potential reaction inducing interaction associated with the corresponding dominant emotion state is performed together with the driver. In detail, referring to FIG. 1A, a specific action associated with the dominant emotion state is requested to be performed (S140). Thereafter, referring to FIG. 1A, a driver feedback responsive to the request is received and discriminated (S150).

In addition, when a dominant emotion state is determined to not exist as a result of determining whether the dominant emotion state exists and thus a reserved dominant emotion state is determined (S160), an explicit/potential reaction inducing interaction associated with the corresponding reserved dominant emotion state is performed. In detail, referring to FIG. 1B, a specific action associated with the reserved dominant emotion state is requested to be performed (S170). Thereafter, referring to FIG. 1B, a driver feedback is received and discriminated (S180).

The method of recognizing the emotion of the driver according to the embodiment of the present invention determines whether the driver feedback is positive or negative through the process of receiving and discriminating the driver feedback based on the reaction inducing interaction. When the driver feedback is determined to be positive (when the driver immediately performs the requested specific action), the weight of the reaction inducing interaction is adjusted upward.

That is, the process of identifying the dominant/reserved emotion of the driver through the emotion recognition information and the process of selecting a drive control-related specific action with respect to the identified emotion and informing the driver (transmission, notification, and the like) are determined to be appropriated, and such processes are assigned an upward-adjusted weight (in other words, when a reaction of the driver with respect to a reaction inducing interaction is positive, it is determined that the procedure for emotion recognition input-to-output and output-to-interaction is desirable, and thus the weight of the procedure is adjusted upward.)

When the driver feedback is determined to be negative (for example, when the driver does not perform the specific action as requested), the weight of the corresponding reaction inducing interaction may be adjusted downward. That is, the process of identifying the dominant/reserved emotion of the driver through the emotion recognition information and the process of selecting a drive control-related specific action with respect to the identified emotion and informing the driver (transmission, notification, and the like) are determined to be inappropriate, and such processes are assigned a downward-adjusted weight (in other words, when a reaction of the driver with respect to a reaction inducing interaction is negative, it is determined that the procedure for emotion recognition input-to-output and output-to-interaction is not desirable, and thus the weight of the procedure is adjusted downward.)

In addition, when the driver feedback is negative, it may be determined whether the process of identifying the dominant/reserved emotion state of the driver through the emotion recognition information is erroneous (an error in the result of the emotion recognition), or whether the process of selecting a drive control-related specific action with respect to the identified emotion state and informing the driver is erroneous (an error in the reaction inducing interaction.) In this case, when a dominant emotion has been derived as a result of a process of recognizing a dominant emotion state through the emotion state recognition process, the process of selecting a specific action in the reaction inducing interaction is determined to be erroneous and requiring a correction. When a reserved dominant emotion state has been derived as a result of a process of recognizing a dominant emotion state through the emotion state recognition process, the process of deriving the reserved dominant emotion state is determined to be erroneous and requiring a correction.

Referring to FIG. 1A, through the above-described procedure, a need to update the emotion recognition model may be determined (S190). In detail, when the weight of a specific process of the reaction inducing interaction is changed through a driver feedback, the changed weight is applied when performing the reaction inducing interaction with the corresponding driver. When the same reaction inducing interaction is performed on the corresponding driver through the subsequent process, the changed weight is applied when the reaction inducing interaction is performed. In other words, when the weight is adjusted upward due to a positive driver feedback, the corresponding reaction inducing interaction is performed in the same manner in the subsequent emotion recognition process, but when a downward adjustment of the weight due to a negative driver feedback is repeated, the corresponding reaction inducing interaction is considered inappropriate.

Accordingly, referring to FIG. 1A, the method of recognizing the emotion of the driver according to the embodiment of the present invention updates the emotion recognition model when the weight is repeatedly adjusted downward due to the negative driver feedback and exceeds a predetermined reference value (a threshold value) (S195). When the degree to which the weight is adjusted downward is not sufficient to update the emotion recognition model, the reaction inducing interaction with the driver is performed using the existing emotion recognition model.

In addition, when an upward adjustment of the weight due to a positive driver feedback from the reaction inducing interaction with respect to the reserved dominant emotion state is performed such that the weight is greater than or equal to a predetermined reference value (a threshold value), the corresponding reserved dominant emotion is converted to a dominant emotion state, and in order to reflect the conversion, the emotion recognition model is updated (S195). Hereinafter, an apparatus for recognizing an emotion of a driver according to an embodiment of the present invention will be described.

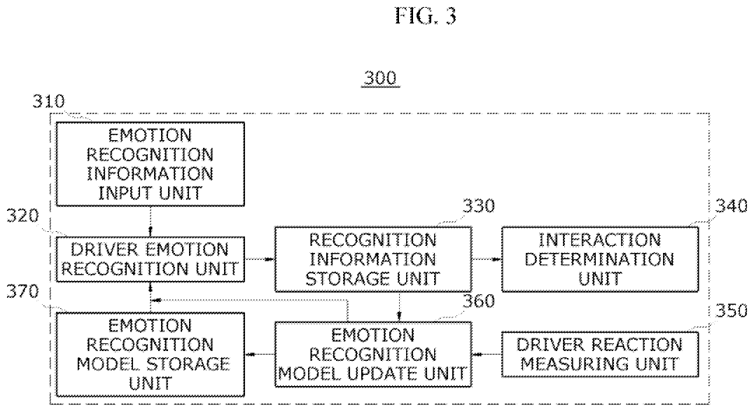

FIG. 3 is a block diagram for describing an apparatus for recognizing an emotion of a driver according to an embodiment of the present invention.

FIG. 3 shows an apparatus 300 for recognizing an emotion of a driver according to the embodiment of the present invention performing the method of recognizing the emotion of the driver shown in FIG. 1. An emotion recognition information input unit 310 receives vehicle state information and driver emotion state information. The driver emotion state information may be measured using sensors installed in the vehicle (a camera, an image sensor, a temperature sensor, and the like.)

The vehicle state information is information related to the driving of the vehicle such as a speed, a degree of acceleration/deceleration, a rotation state, sudden acceleration, internal and external lighting conditions, and the noise state of the vehicle. Such vehicle state information may be measured from various sensors installed in the vehicle or received through an in-vehicle device such as an ECU.

A driver emotion recognition unit 320 determines an existence of a dominant emotion state and an existence of a reserved dominant emotion state of the driver on the basis of the received emotion recognition information and an emotion recognition model and generates at least one of dominant emotion state information and reserved dominant emotion state information according to the determination.

An emotion recognition model storage unit 370 stores an emotion recognition model of the driver. The emotion recognition mode of the driver represents an emotion of the driver identified from a facial expression, dialogue content, an action (including a gesture), and various biosignals of the driver. That is, the emotion recognition model of the driver represents a relationship between an identified specific facial expression, dialog content, dialog speed, and specific gesture, and a specific emotion state of the driver. According to an update result of an emotion recognition model update unit 360, the stored emotion recognition model may be changed and stored in the emotion recognition model storage unit 370.

An interaction determination unit 340 requests the driver to perform a drive control-related specific action on the basis of at least one of the generated dominant emotion state information and the generated reserved dominant emotion state information.

A driver reaction measuring unit 350 receives driver feedback information from the driver in response to the request sent by the interaction determination unit 340 for performing the specific action. The driver reaction measuring unit 350 may analyze and discriminate the received driver feedback information and adjust the weight of the reaction inducing interaction.

When the driver feedback information is determined to be positive (when the driver immediately performs the requested specific action), the weight of the corresponding reaction inducing interaction is adjusted upward. The adjusting of the weight upward includes performing at least one of an operation of adjusting the weight of the generation of the dominant emotion state information upward by the driver emotion recognition unit 320 and/or an operation of adjusting the weight of the request for the performance of a specific action upward by the interaction determination unit 340.

When the driver feedback information is determined to be negative (when the driver does not perform the requested specific action), the weight of the corresponding reaction inducing interaction may be adjusted downward. The adjusting of the weight downward includes performing at least one of an operation of adjusting the weight of the generation of the reserved dominant emotion state information downward by the driver emotion recognition unit 320 and/or an operation of adjusting the weight of the request for a specific action downward by the interaction determination unit 340.

In addition, when the driver feedback is negative, it may be determined whether the process of identifying the dominant/reserved emotion state of the driver through the emotion recognition information is erroneous (an error in the result of the emotion recognition), or whether the process of selecting a drive control-related specific action with respect to the identified emotion state and informing the driver is erroneous (an error in the reaction inducing interaction.)

In this case, when a dominant emotion has been derived as a result of a process of recognizing a dominant emotion state through the emotion state recognition process, the process of selecting a specific action in the reaction inducing interaction is determined to be erroneous and requiring a correction (a need to change the request for performing the drive control-related specific action with respect to the dominant emotion state of the driver.) When a reserved dominant emotion state has been derived as a result of a process of recognizing a dominant emotion state through the emotion state recognition process, the process of deriving the reserved dominant emotion state is determined to be erroneous and requiring a correction (a need to update the reserved dominant emotion state information.)

The emotion recognition model update unit 360 may update the emotion recognition model that is used for the reaction inducing interaction on the basis of the driver feedback from the driver reaction measuring unit 350.

When the weight is repeatedly adjusted downward due to the negative driver feedback and exceeds a predetermined reference value (a threshold value), the emotion recognition model may be updated. In addition, when an upward adjustment of the weight due to a positive driver feedback in the reaction inducing interaction with respect to the reserved dominant emotion state is performed such that the weight is greater than or equal to a predetermined reference value (a threshold value), the corresponding reserved dominant emotion is converted to a dominant emotion state, and in order to reflect the conversion of the dominant emotion state, the emotion recognition model may be updated.

Meanwhile, the above-described components have been described as separated devices but are to be regarded as illustrative to aid in the explanation and understanding of the present invention and may be implemented in various forms within the scope and spirit of the present invention. For example, a recognition information storage unit 330 and the emotion recognition model storage unit 370 may be implemented as separated devices or as an integrated single device.

The methods according to the present invention may be implemented in the computer system. The computer system may include at least one of processor, memory, user input device, data communication bus, user output device, and storage. Each component of the computer system communicates with the data commnication bus.

The methods according to the present invention may be implemented in the form of program commands executable by various computer devices and may be recorded in a computer readable media. The computer readable media may be provided with program commands, data files, data structures, and the like alone or in a combination thereof. The program commands stored in the computer readable media may be those specially designed and constructed for the purposes or may be of the kind well-known and available to those having skill in the computer software arts.

Examples of the computer readable storage medium include a hardware device constructed to store and execute a program command, for example, a read only memory (ROM), a random access memory (RAM), and a flash memory. The program command may include a high-level language code executable by a computer through an interpreter in addition to a machine language code made by a compiler. The described hardware devices may be configured to act as one or more software modules in order to perform the operations of the present invention, or vice versa.

As is apparent from the above, the accuracy in identifying the emotion state of the driver can be enhanced through a driver-to-vehicle reaction inducing interaction check process based on an analysis of emotion state information of the driver and vehicle state information.

Although the constructions of the present invention have been described in detail with reference to the embodiments, the above embodiments should be regarded as illustrative rather than limitative in all aspects. A person of ordinary skill in the art should appreciate that various modifications and equivalents derived from the teaching and suggestion of the above specification fall within the scope and sprit of the present invention. Therefore, the scope of the present invention is defined by the appended claims of the present invention.

What is claimed is:

1. A method of recognizing an emotion of a driver, the method comprising:
receiving emotion recognition information including at least one of vehicle state information and driver emotion state information;
determining whether a dominant emotion state exists among one or more emotion states possessed by the driver on the basis of the emotion recognition information and an emotion recognition model; and
performing a reaction inducing interaction with respect to the driver determined on the basis of the determined dominant emotion state together with the driver when the dominant emotion state of the driver is determined to exist.

2. The method of claim 1, wherein the determining of whether the dominant emotion state exists includes:
identifying the number of emotion states possessed by the driver from the emotion recognition information;
converting the identified emotion states into dominant emotion indexes;
identifying whether a dominant emotion index exceeding a predetermined threshold value among the dominant emotion indexes exists; and
in response to an existence of the dominant emotion indexes exceeding a predetermined reference value, determining an emotion state corresponding to a dominant emotion index exceeding the predetermined reference value to a largest degree to be the dominant emotion state.

3. The method of claim 1, wherein the performing of the reaction inducing interaction with respect to the driver together with the driver includes:
requesting the driver to perform a specific action related to a drive control;
receiving driver feedback information responsive to the request for performing the specific action from the driver;
adjusting a weight of the reaction inducing interaction upward when the received driver feedback information is determined to be a positive response; and
adjusting a weight of the reaction inducing interaction downward when the received driver feedback information is determined to be a negative response.

4. The method of claim 3, wherein the adjusting of the reaction inducing interaction upward includes at least one of:
adjusting a weight of a conversion of a dominant emotion index upward in the determining of whether the dominant emotion state exists; and
adjusting a weight of the request for the performance of the specific action upward in the performing of the reaction inducing interaction with the driver.

5. The method of claim 3, wherein the adjusting of the reaction inducing interaction downward includes at least one of:
adjusting a weight of a conversion of a dominant emotion index downward in the determining of whether the dominant emotion state exists; and
adjusting a weight of the request for the performance of the specific action downward in the performing of the reaction inducing interaction with the driver.

6. The method of claim 2, further comprising, when the dominant emotion state of the driver is determined to not exist,
selecting N emotion states starting from an emotion state having a highest dominant emotion index first among the one or more emotion states derived in the determining of whether the dominant emotion state of the driver exists;
determining an emotion state randomly selected from the N emotion states to be a reserved dominant emotion state; and
performing a reaction inducing interaction with respect to the driver determined on the basis of information about the determined reserved dominant emotion state together with the driver.

7. The method of claim 6, wherein the adjusting of a weight of the reaction inducing interaction downward includes:
changing the request for performing the specific action with respect to the dominant emotion state of the driver when the dominant emotion state of the driver is determined to exist, and the driver feedback information indicates a negative response; and
updating the determined reserved dominant emotion state when the dominant emotion state of the driver is determined to not exist, and the driver feedback information indicates a negative response.

8. An apparatus for recognizing an emotion of a driver, the apparatus comprising:
an emotion recognition information input unit configured to receive emotion recognition information including at least one of vehicle state information and driver emotion state information;
an emotion recognition model storage unit configured to store an emotion recognition model of the driver;
a driver emotion recognition unit configured to generate at least one of dominant emotion state information and reserved dominant emotion state information of the driver on the basis of the received emotion recognition information and the emotion recognition model;
an interaction determination unit configured to request the driver to perform a specific action related to a drive control on the basis of the determined at least one of the dominant emotion state information and the reserved dominant emotion state information of the driver;
a driver response measurement unit configured to receive driver feedback information responsive to the request for performing the specific action from the driver; and
a recognition information storage unit configured to store at least one of the emotion recognition information of the driver, the dominant emotion state information of the driver, and the reserved dominant emotion state information of the driver.

9. The apparatus of claim 8, further comprising an emotion recognition model update unit configured to perform an update on the emotion recognition model on the basis of information about updating the emotion recognition model of the driver that is generated on the basis of the driver feedback information.

10. The apparatus of claim 8, wherein the driver emotion recognition unit is configured to:
identify the number of emotion states possessed by the driver from the emotion recognition information;
convert the identified emotion states into dominant emotion indexes;
identify whether a dominant emotion index exists that exceeds a predetermined threshold value among the dominant emotion indexes; and
in response to an existence of the dominant emotion indexes exceeding a predetermined reference value, generate an emotion state corresponding to a dominant emotion index exceeding the predetermined reference value to a largest degree as dominant emotion state information.

11. The apparatus of claim 8, wherein the driver emotion recognition unit is further configured to:
identify the number of emotion states possessed by the driver from the emotion recognition information;
convert the identified emotion states into dominant emotion indexes;
identify whether a dominant emotion index exists that exceeds a predetermined threshold value among the dominant emotion indexes ;
sequentially select N dominant emotion indexes starting from a largest dominant emotion index first among the dominant emotion indexes in response to an absence of the dominant emotion index exceeding a predetermined reference value; and
generate an emotion state associated with a dominant emotion index randomly selected among the N dominant emotion indexes as reserved dominant emotion state information.

12. The apparatus of claim 8, wherein the driver response measurement unit adjusts a weight of the request sent to the driver for the performance of the specific action related to the drive control upward when the received driver feedback information is determined to be a positive response,
wherein the adjusting of the weight upward includes at least one of:
adjusting a weight of the generation of the dominant emotion state information performed by the driver emotion recognition unit upward; and
adjusting a weight of the request for the performance of the specific action sent by the interaction determination unit upward.

13. The apparatus of claim 8, wherein the driver response measurement unit adjusts a weight of the request sent to the driver for the performance of the specific action related to the drive control downward when the received driver feedback information is determined to be a negative response,
wherein the adjusting of the weight downward includes at least one of:
adjusting a weight of the generation of the reserved dominant emotion state information performed by the driver emotion recognition unit downward; and
adjusting a weight of the request for the performance of the specific action sent by the interaction determination unit downward.

14. The apparatus of claim 13, wherein the downward adjusting of the weight of the request for the performance of the specific action sent by the interaction determination unit includes:
when the driver response measurement unit receives negative driver feedback information in response to a request sent by the interaction determination unit to the driver for performing a drive-control related specific action based on the dominant emotion state information, changing the request for performing the drive-control related specific action associated with the dominant emotion state information of the driver; and
when the driver response measurement unit receives negative driver feedback information in response to a request sent by the interaction determination unit to the driver for performing a drive-control related specific action based on the reserved dominant emotion state information, updating the generated reserved dominant emotion state information.

15. A method of recognizing an emotion of a driver, the method comprising:
receiving emotion recognition information including at least one of vehicle state information and driver emotion state information;
deriving at least one emotion state of the driver included in the emotion recognition information;
converting the derived emotion states of the driver into dominant emotion indexes;
arranging the converted dominant emotion indexes in an order of magnitude; and
determining whether a dominant emotion exists by comparing the arranged dominant emotion indexes with a reference value for determining the dominant emotion.

16. The method of claim 15, wherein the determining of whether the dominant emotion exists includes:
deriving a dominant emotion index larger than the reference value; and
in response to an existence of one or more dominant emotion indexes larger than the reference value, selecting a largest dominant emotion index and determining a dominant emotion state associated with the largest dominant emotion index to be a dominant emotion state.

17. The method of claim 15, wherein the determining of whether the dominant emotion exists includes:
deriving a dominant emotion index larger than the reference value;
sequentially selecting N dominant emotion indexes starting from a largest dominant emotion index first in response to an absence of the dominant emotion index larger than the reference value; and
determining an emotion state associated with a dominant emotion index randomly selected among the N dominant emotion indexes to be a reserved dominant emotion state.
